(19) United States
(12) Patent Application Publication
(10) Pub. No.: US 2026/0055134 A1
(43) Pub. Date: Feb. 26, 2026

(54) SUSTAINABLE METHOD FOR SOLID-PHASE PEPTIDE SYNTHESIS

(71) Applicant: AAPPTec, LLC, Louisville, KY (US)

(72) Inventors: Hossain Saneii, Prospect, KY (US); Rajan Sharma, Louisville, KY (US); Fatemeh Karimi Tabar, Louisville, KY (US)

(21) Appl. No.: 19/307,978

(22) Filed: Aug. 22, 2025

Related U.S. Application Data
(60) Provisional application No. 63/685,937, filed on Aug. 22, 2024.

(51) Int. Cl. C07K 1/06; C07K 1/04

(52) U.S. Cl. CPC C07K 1/063; C07K 1/042; C07K 1/064

(57) ABSTRACT

Provided herein is an environmentally friendly method of solid-phase peptide synthesis (SPPS), wherein the method employs isopropyl alcohol (IPA) as the major solvent or co-solvent in the synthesis steps of deprotection, washing, and coupling. The presently disclosed methods employ IPA in place of traditional solvents such as N,N-dimethylformamide (DMF) and dichloromethane (DCM).

### CROSS-REFERENCE TO RELATED APPLICATION

This application claims priority to U.S. Provisional Application Ser. No. 63/685,937, filed Aug. 22, 2024, the entire contents of which are incorporated herein by reference.

### TECHNICAL FIELD

The present disclosure relates to a method for synthesizing peptides. More specifically, the present disclosure relates to an improved, environmentally sustainable method for solid phase peptide synthesis (SPPS).

### SEQUENCE LISTING

A sequence listing in XML format, having file name AAX0002PA_Sequence.xml, created on Aug. 22, 2025 (15,583 bytes) is incorporated herein by reference.

### BACKGROUND

Peptides are essential building blocks in living organisms, consisting of short chains of amino acids linked by peptide bonds. Therapeutic peptides offer several advantages in the pharmaceutical field. They are easier to manufacture compared to full-length proteins and exhibit higher selectivity in their activities. Additionally, peptides may provoke fewer immune responses, making them valuable in clinical settings for treating various diseases and conditions.

The importance of peptides in pharmaceutical applications has significantly increased in recent years, with the emergence of new peptides in the market. These include medium-sized peptides with cyclic or bicyclic structures, as well as large peptides incorporating unnatural amino acids and non-peptide components such as fatty acids or polyethylene glycol chains.

Solid-phase peptide synthesis (SPPS) is a method for creating peptides by assembling amino acids stepwise on a solid support, such as a resin. SPPS offers advantages in automated synthesis for high-throughput drug discovery. However, standard methods for SPPS have significant drawbacks. For example, one metric for assessing efficiency of a chemical process is determining the process mass intensity (PMI). PMI is calculated as the total mass of all input materials divided by the mass of the final product. The lower the PMI, the more efficient and sustainable a process is considered. Conventional SPPS is not only expensive from a materials perspective, but the PMI is high compared to solution chemistry, averaging around 13,000. Moreover, commonly used organic solvents used in SPPS, such as N,N-dimethylformamide (DMF) and dichloroethane (DCM), are not environmentally friendly.

Despite numerous efforts by academic and industrial groups, efforts to replace DMF with a single solvent have been unsuccessful. N-butyl-2-pyrrolidone (NBP) has shown promise as an alternative, but its high viscosity affects performance, impacting resin swelling and presenting challenges to solvent transfer and automation. Binary solvent mixtures, such as ethyl acetate (EtOAc) with dimethylsulfoxide (DMSO) or NBP, have also been explored.

A need exists for an efficient, environmentally friendly, “green” solvent to replace DMF in methods of SPPS.

### SUMMARY

It has now been found that isopropyl alcohol (IPA) can advantageously be used as a major solvent or co-solvent in methods of SPPS. IPA is a cost effective, environmentally friendly alternative to solvents such as DMF or DCM that yields high quality synthesized peptides.

In one embodiment, a method of solid-phase peptide synthesis (SPPS) of a peptide is provided, the method comprising:
(a) coupling a protected amino acid to a resin;
(b) contacting the resin with a solvent comprising isopropyl alcohol (IPA) to swell the resin;
(c) deprotecting the protected amino acid with a deprotection solution comprising IPA to provide a deprotected terminal amino acid;
(d) washing the product of step (c) with a washing solution comprising IPA;
(e) coupling an activated amino acid to the deprotected amino acid in the presence of a coupling solution comprising IPA;
(f) washing the product of step (e) with the washing solution;
(g) washing the product of step (f) with methanol, methyl-tert-butyl-ether (MTBE), or diethyl ether.

In another embodiment, a method of solid phase peptide synthesis (SPPS) of a peptide is provided, wherein IPA is a major solvent or a co-solvent in all washing steps, and wherein the method does not employ N,N-dimethylformamide (DMF) as a solvent in any step.

These and other features, aspects, and advantages will become better understood with reference to the following description and the appended claims.

Additional features and advantages of the embodiments described herein will be set forth in the detailed description that follows, and in part will be readily apparent to those skilled in the art from that description or recognized by practicing the embodiments described herein, including the detailed description that follows, the claims, as well as the appended drawings.

### BRIEF DESCRIPTION OF THE DRAWINGS

FIG. 1 is a flow chart of a method of solid-phase peptide synthesis, according to one or more embodiments of the present disclosure.

FIG. 2 is a graph depicting the HPLC analysis of the crude of Peptide 1.

FIG. 3 is a graph depicting the HPLC analysis of the crude of Peptide 2.

FIG. 4 is a graph depicting the HPLC analysis of the crude of Peptide 3.

FIG. 5 is a graph depicting the HPLC analysis of the crude of Peptide 4.

FIG. 6 is a graph depicting the HPLC analysis of the crude of Peptide 5.

FIG. 7 is a graph depicting the HPLC analysis of the crude of Peptide 6.

FIG. 8 is a graph depicting the HPLC analysis of the crude of Peptide 7.

FIG. 9 is a graph depicting the HPLC analysis of the crude of Peptide 8.

FIG. 10 depicts mass of the crude of Peptide 8.

FIG. 11 is a graph depicting the HPLC analysis of the crude of Peptide 9.

FIG. 12 depicts mass of the crude of Peptide 9.

FIG. 13 is a graph depicting the HPLC analysis of the crude of Peptide 10.

FIG. 14 is a graph depicting the HPLC analysis of the crude of Peptide 11.

FIG. 15 is a graph depicting the HPLC analysis of the crude of Peptide 12.

FIG. 16 is a graph depicting the HPLC analysis of the crude of Peptide 13.

FIG. 17 is a graph depicting the HPLC analysis of the crude of Peptide 14.

FIG. 18 is a graph depicting the HPLC analysis of the crude of Peptide 15.

FIG. 19 is a graph depicting the HPLC analysis of the crude of Peptide 16.

FIG. 20 is a graph depicting the HPLC analysis of the crude of Peptide 17.

### DETAILED DESCRIPTION

The details of embodiments of the presently-disclosed subject matter are set forth in this document. Modifications to embodiments described in this document, and other embodiments, will be evident to those of ordinary skill in the art after a study of the information provided in this document.

While the following terms are believed to be well understood in the art, definitions are set forth to facilitate explanation of the presently-disclosed subject matter. Unless defined otherwise, all technical and scientific terms used herein have the same meaning as commonly understood by one of ordinary skill in the art to which the presently-disclosed subject matter belongs.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

As used in this specification and the appended claims, the singular forms “a,” “an” and “the” include plural references unless the content clearly dictates otherwise.

Solid-phase peptide synthesis (SPPS) has long been the preferred method for synthesizing diverse peptide sequences, but its extensive solvent use poses environmental challenges. The present disclosure provides an environmentally friendly, “green” approach to peptide synthesis that replaces conventional solvents with isopropyl alcohol (IPA). IPA offers a sustainable alternative, addressing the negative environmental footprint associated with peptide chemistry. The present disclosure evidences the successful implementation of IPA as a major solvent or co-solvent in methods of SPPS. The presently disclosed methods are suitable for automation, scalability, and largescale peptide production of peptides, including therapeutic peptides, such as popular GLP analogs including exenatide, liraglutide, semaglutide, and tirzepatide. By adopting an IPA-based SPPS approach, peptide synthesis can be conducted in a more environmentally responsible manner, aligning with global efforts to promote sustainable practices in pharmaceutical manufacturing.

The desired characteristics of a green solvent for replacing N,N-dimethylformamide (DMF) as a major solvent in SPPS, in conjunction with polystyrene-based resins, include a melting point ≤10° C., a boiling point ≥80° C., a viscosity <4 mPa s, solubility of fluorenylmethylaminoacids (Fmoc) and coupling reagents, as well as by-products, ≥0.25 M, starting resin swelling of 4-7 mL/g, coupling time <1 hour, Fmoc-removal time <30 minutes, and cost effectiveness.

IPA offers several advantages as a major solvent or co-solvent for SPPS. IPA is considered a safe solvent with low toxicity, aligning with its common use in laboratories. IPA is regarded as a green solvent due to its biodegradability and low environmental impact. IPA is easily accessible, commercially available, and cost effective compared to other solvents commonly used in peptide synthesis. Disposing of IPA is relatively easy and inexpensive, contributing to the overall efficiency of the peptide synthesis process. Further, IPA exhibits good solubility for most amino acids, making it suitable for dissolving and handling amino acids during peptide synthesis. IPA has a better flash point compared to certain other solvents, enhancing safety during handling and storage. IPA is also compatible with a wide range of other solvents, enabling its use in various solvent mixtures for specific peptide synthesis steps. Additionally, 100% IPA can cause resin shrinkage, which can be advantageous in certain steps of peptide synthesis, whereby the reduced need for multiple washes results in significant time and cost savings during peptide synthesis. IPA is a versatile solvent that can effectively dissolve amino acids, reagents, and other protecting groups involved in SPPS. Table 1 sets forth the solubilities of amino acids (0.2 M/ml) in IPA 100%, IPA/DMSO (70:30), and IPA/DMSO (40:60).

TABLE 1
Solubility of Amino Acids (0.2M/ml) in IPA*

| | IPA | IPA/DMSO | IPA/DMSO |
|---|---|---|---|
| Amino Acid | (100%) | (70:30)* | (40:60)* |
| Fmoc-Ala-OH | No | Yes | Yes |
| Fmoc-Glu(tBu)-OH | Yes | Yes | Yes |
| Fmoc-Gly-OH | No | Yes | Yes |
| Fmoc-Ile-OH | No | Yes | Yes |
| Fmoc-Leu-OH | No | Yes | Yes |
| Fmoc-Lys(Boc)-OH | No | Yes | Yes |
| Fmoc-Met-OH | No | Yes | Yes |
| Fmoc-Thr(tBu)-OH | Yes | Yes | Yes |
| Fmoc-Ser(tBu)-OH | Yes | Yes | Yes |
| Fmoc-Phe-OH | No | Yes | Yes |
| Fmoc-Pro-OH | Yes | Yes | Yes |
| Fmoc-Tyr(tBu)-OH | No | Yes | Yes |
| Fmoc-Gln(Trt)-OH | No | Yes | Yes |
| Fmoc-Val-OH | No | Yes | Yes |
| Fmoc-Asp(tBu)-OH | No | Yes | Yes |
| Fmoc-Trp(Boc)-OH | No | Yes | Yes |
| Fmoc-Arg(pbf)-OH | No | No | Yes |
| Fmoc-Asn(Trt)-OH | No | No | Yes |
| Fmoc-Cys(Trt)-OH | No | No | Yes |
| Fmoc-His(Trt)-OH | No | No | Yes |

*Depending on the protected amino acid production in powder form, elevated temperature (40° C.) may be necessary to dissolve the amino acids. Once solubilized, these amino acids can remain in solution at room temperature.

Table 2 sets forth the solubilities of amino acids (0.3 M/ml) in IPA 100%, IPA/DMSO (70:30), and IPA/DMSO (40:60).

TABLE 2
Solubility of Amino Acids (0.3M/ml) in IPA*

| | IPA | IPA/DMSO | IPA/DMSO |
|---|---|---|---|
| Amino Acid | (100%) | (70:30)* | (40:60)* |
| Fmoc-Ala-OH | No | Yes | Yes |
| Fmoc-Glu(tBu)-OH | Yes | Yes | Yes |
| Fmoc-Gly-OH | No | Yes | Yes |
| Fmoc-Ile-OH | No | Yes | Yes |
| Fmoc-Leu-OH | No | Yes | Yes |
| Fmoc-Lys(Boc)-OH | No | Yes | Yes |
| Fmoc-Met-OH | No | Yes | Yes |
| Fmoc-Thr(tBu)-OH | Yes | Yes | Yes |
| Fmoc-Ser(tBu)-OH | Yes | Yes | Yes |
| Fmoc-Phe-OH | No | Yes | Yes |
| Fmoc-Pro-OH | Yes | Yes | Yes |
| Fmoc-Tyr(tBu)-OH | No | Yes | Yes |
| Fmoc-Gln(Trt)-OH | No | Yes | Yes |
| Fmoc-Val-OH | No | Yes | Yes |
| Fmoc-Asp(tBu)-OH | No | Yes | Yes |
| Fmoc-Trp(Boc)-OH | No | Yes | Yes |
| Fmoc-Arg(pbf)-OH | No | No | Yes |
| Fmoc-Asn(Trt)-OH | No | No | Yes |
| Fmoc-Cys(Trt)-OH | No | No | Yes |
| Fmoc-His(Trt)-OH | No | No | Yes |

*Depending on the protected amino acid production in powder form, elevated temperature (40° C.) may be necessary to dissolve the amino acids. Once solubilized, these amino acids can remain in solution at room temperature.

Referring to SPPS processes, the term “major solvent” refers to the primary solvent used throughout the synthesis process. The major solvent may be used in resin swelling, dissolving amino acids, coupling reactions (forming peptide bonds), deprotection reactions (removing protecting groups), and washing steps. Traditionally, SPPS has used polar aprotic solvents such as DMF and DCM as major solvents. In embodiments, it may be preferable to use solvent mixtures, or co-solvents, to optimize synthetic parameters. The disclosed methods use IPA as a major solvent or a component of a co-solvent or solvent solution for steps including resin swelling, dissolving amino acids, coupling reactions, deprotection reactions, and washing steps.

In certain steps of the methods disclosed herein, IPA is combined with DMSO or ethyl acetate (EtOAc) to provide a co-solvent mixture or solvent mixture. For example, the solvent mixture DMSO-IPA (30:70), may be used for swelling and washing the resin during various steps of peptide synthesis, or during deprotection and coupling steps. The IPA solvent mixture efficiently swells the resin, facilitating the removal of various protecting groups (Table 3). In traditional synthetic process of SPPS, Fmoc, and Boc are commonly used as α-amino protecting groups, as well as specific protecting groups for side chains of amino acids. By way of non-limiting examples, tBu may be used as a protecting group for the side chains of Ser, Thr, Tyr, Asp, and Glu; Boc may be used as a protecting group for the side chains of Lys, Trp, and His; Trt may be used as a protecting group for the side chains of Asn, Gln, Cys, and His; Pbf may be used as a protecting group for the side chains of Arg; Acm may be used as a protecting group for the side chains of Cys, and others with similar performance. IPA, IPA-DMSO, and IPA-EtOAc are suitable for use in resin swelling and facilitating removal of any such protecting groups.

IPA and IPA solvent mixtures are suitable for use with a variety of resins commonly used in SPPS. Suitable resins may optionally comprise linkers for binding the terminal amino acid. Suitable resins may include polystyrene-based resins, AAPPTec resin, Oxime resin, Wang resin, MBHA resin, Rink Amide resin, trityl based resins, Sieber resin, PAM (phenylacetamidomethyl) resin, PEG-based resins, (e.g., TentaGel®, Octagel™), and polyacrylamide resins (e.g., Amino-Li resin). Resins may further comprise linkers, which may include Wang linker, PAL, Rink Amide linker, 2-chlorotrityl linker, HMBA/HMPA linkers, HMBP linkers, PAM linkers, Merrifield linker, safety catch linkers, Rink Amide-AM, Rink Amide-MBHA, Sieber linker, Ramage linker, and the like. It is within the purview of the ordinary skilled person to select the appropriate resin/linker for synthesizing peptides according to the present methods.

TABLE 3
Swelling of Resin 200 mg/2 ml in IPA

| Resin | Resin & solvent | Height in mm | |
|---|---|---|---|
| OctaGel ™ Rink | Only Resin | 7 | mm |
| amide resin | DMF | 20 | mm |
| | IPA | 10 | mm |
| | 30% DMSO in IPA | 17 | mm |
| | 50% DMSO in IPA | 18 | mm |
| Rink amide AM | Only Resin | 5.5 | mm |
| resin | DMF | 17 | mm |
| | IPA | 7 | mm |
| | 30% DMSO in IPA | 8 | mm |
| | 50% DMSO in IPA | 14 | mm |

IPA exhibits favorable solvating properties for many coupling reagents commonly employed in SPPS (Table 4). Advantageously, it has now been found that IPA enables efficient and reliable peptide bond formation during the coupling steps, ensuring high yields and purity of the synthesized peptides.

TABLE 4
Solubility of Coupling Reagents in IPA

| Reagents | IPA (100%) | 50% IPA in DMSO | 100% DMSO |
|---|---|---|---|
| DIC | Yes | Yes | Yes |
| HBTU | No | No | Yes |
| HATU | No | No | Yes |
| TBTU | No | No | Yes |
| PyBOP | No | Yes | Yes |
| PyAOP | No | Yes | Yes |
| COMU | No | Yes | Yes |
| BOP | No | Yes | Yes |
| BOP•Cl | No | No | Yes |
| Cl-HOBT | No | Yes | Yes |
| EDC•HCl | Yes | Yes | Yes |
| Oxyma | Yes | Yes | Yes |

*To use the above-referenced reagents for coupling, the reagent may be solubilized in DMSO and then diluted with amino acids in IPA or IPA in DMSO. They will remain in the solution form for further use.

The presently disclosed methods permit production of peptides ranging from small (2-50 amino acid residues) to large sizes (50-100 residues), including GIP/GLP1 dual agonist peptides tirzepatide, exenatide, semaglutide, liraglutide, as well as acyl carrier protein (ACP), angiotensin, and the like, utilizing automated and microwave techniques, or their pharmaceutically acceptable salts. The disclosed methods involve more stable intermediates to provide larger peptides with fewer purification steps.

SPPS involves coupling an activated amino acid (usually the terminal or last amino acid in the sequence) to a solid support. This solid support may be a polymeric resin bead that is functionalized (such as with an NH₂ group). The terminal amino acid (typically protected at its NH₂ terminus via a Fmoc, Boc or other suitable protecting group) is reacted with the resin such that the functionalized group on the resin reacts with and binds to the activated COOH group of the terminal amino acid. In this manner, the terminal amino acid is covalently attached to the resin. Activated amino acids are then added to the growing amino acid chain, stepwise through repeated rounds of deprotection, washing, and coupling.

The disclosed methods provide novel intermediates and processes useful in the manufacture of peptides, or a pharmaceutically acceptable salt thereof. The improved manufacturing processes provide intermediates and process reactions embodying a combination of advances, including an efficient protocol having fewer steps, while at the same time maintaining high quality and purity of synthesized peptide products. Importantly, the improved processes and intermediates decrease resource intensity and minimize waste streams, providing an attractive green alternative to traditional SPPS methods.

### Methods of SPPS

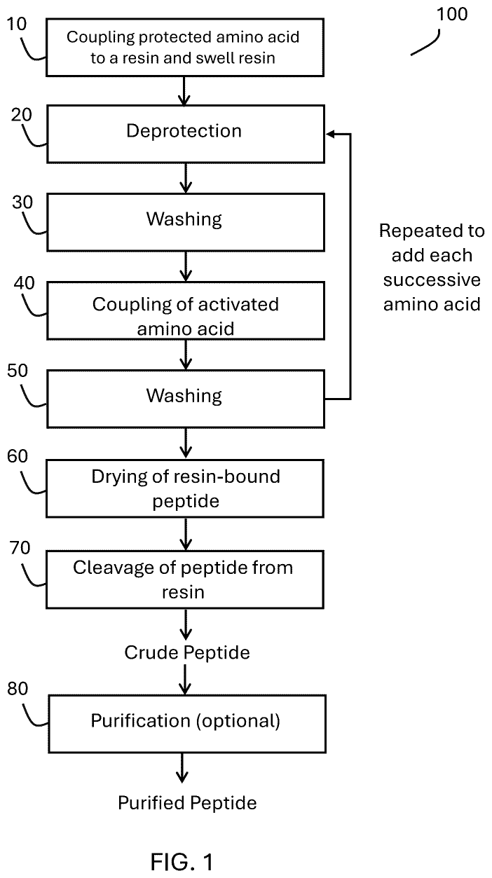

FIG. 1 is a flow chart depicting an embodiment of an exemplary SPPS method 100 according to the present disclosure. At block 10, a protected amino acid, typically a terminal amino acid, is coupled to a resin. The resin is then contacted with a solvent comprising IPA and optionally IPA in combination with DMSO to swell the resin. At block 20, the protected amino acid bound to the resin is deprotected with a deprotection solution comprising IPA, to provide a deprotected resin-bound amino acid. At block 30, the deprotected resin-bound amino acid is washed with a washing solution comprising IPA and optionally DMSO or EtOAc. At block 40, an activated amino acid is coupled to the resin-bound deprotected amino acid via a coupling solution comprising IPA and optionally DMSO or EtOAc. At block 50, the growing amino acid chain bound to the resin is washed in the washing solution comprising IPA and optionally DMSO or EtOAc. At this point, blocks 20-50 may be repeated to add sequential amino acids, until the amino acid sequence of the peptide is completed. Once the amino acid sequence is completed, the resin-bound peptide may be washed again with methanol, methyl-tert-butyl-ether (MTBE), or diethyl ether. Next, the process moves to block 60, where the resin-bound peptide is dried. The peptide is cleaved from the resin at block 70 to provide a crude peptide. The crude peptide is then optionally purified at block 80 to provide a purified peptide product.

Accordingly, in one embodiment, a method of solid-phase peptide synthesis (SPPS) of a peptide is provided, the method employing sequential couplings to provide a peptide product. In embodiments, the method comprises:
(a) coupling a protected amino acid to a resin;
(b) contacting the resin with a solvent comprising isopropyl alcohol (IPA) to swell the resin;
(c) deprotecting the protected amino acid with a deprotection solution comprising IPA to provide a deprotected amino acid;
(d) washing the product of step (c) with a washing solution comprising IPA;
(c) coupling an activated amino acid to the deprotected amino acid in the presence of a coupling solution comprising IPA;
(f) washing the product of step (c) with the washing solution comprising IPA;
(g) washing the product of step (f) (the resin-bound peptide) with methanol, methyl-tert-butyl-ether (MTBE), or diethyl ether.

In embodiments, the amino acid of step (a) is a terminal amino acid of the peptide to be synthesized, which is protected with a protecting group at its a-amino group. The terminal amino acid is bound to the resin at its C-terminus, optionally via a linker moiety. In embodiments, the terminal amino acid may also comprise orthogonal protecting groups on reactive side chains. The ordinary skilled person will appreciate that various protecting groups are suitable for use in the presently disclosed methods and are known in the art. Non-limiting examples of suitable protecting groups include 9-fluorenylmethoxycarbonyl (Fmoc), tert-butyloxycarbonyl (Boc), carboxybenzyl (CBZ), 1-(4,4-dimethyl-2,6-dioxocyclohex-1-ylidene)ethyl(Dde), and the like. Additional exemplary protecting groups are set forth in U.S. Pat. Nos. 8,219,327 and 8,309,547, each of which is incorporated by reference in its entirety. In specific embodiments, the terminal amino acid comprises a Fmoc or Boc protecting group.

In embodiments, swelling the resin in step (b) is carried out using IPA, or a solvent solution comprising IPA and dimethylsulfoxide (DMSO). In embodiments, the solvent solution may comprise DMSO at a concentration of from about 30% to about 40% by volume, and may comprise IPA at a concentration of from about 70% to about 60% by volume.

In embodiments, the deprotection solution may comprise IPA, or may comprise a combination of IPA and DMSO or IPA and EtOAc. In embodiments, the deprotection solution employed in the deprotection step (c) further comprises piperidine (Pip) and/or 1,8-Diazabicyclo[5.4. 0]undec-7-ene (DBU). For example, in a specific embodiment, deprotection is carried out using 20% Pip or 20% Pip with 1% DBU in IPA/DMSO, wherein DMSO is present in the solution at a concentration of about 30% or more. Deprotection steps may be carried out at room temperature or under heated conditions.

In embodiments, after deprotection, the resin-bound peptide is washed in step (d) with a washing solution comprising IPA or optionally IPA and one of DMSO or EtOAc. Illustratively, in embodiments, the resin is washed 4-6 times, for ˜2-3 minutes each wash, in 10 volumes of IPA/DMSO, wherein the concentration of DMSO may be about 30% or more. Such washing steps may be carried out at room temperature or under heated conditions.

Amino acid pre-activation may be carried out to convert the carboxylic group of an incoming amino acid into a reactive intermediate and facilitate effective coupling. Methods of amino acid activation are well known in the art. For example, in embodiments, incoming amino acids are activated using DIC/Oxyma or HOBT with IPA/DMSO, wherein the concentration of DMSO may be about 30% and more. This step may be caried out at room temperature or under heated conditions.

The coupling of the activated amino acid to the resin-bound peptide at step (e) is carried out using a coupling solution comprising IPA, or optionally IPA and one of DMSO or EtOAc. Use of a solvent solution such as IPA/DMSO, IPA/EtOAc, or EtOAc is particularly useful for coupling amino acids having low solubility in IPA. In embodiments, the activated amino acid is protected with a protecting group at its a-amino group, and may also comprise orthogonal protecting groups on reactive side chains. Various protecting groups are known and suitable for use in the present methods. Non-liming examples include 9-fluorenylmethoxycarbonyl (Fmoc), tert-butyloxycarbonyl (Boc), carboxybenzyl (CBZ), 1-(4,4-dimethyl-2,6-dioxocyclohex-1-ylidene)ethyl(Dde), and the like. In a specific embodiment, the protecting group is Fmoc or Boc. Coupling steps may be carried out at room temperature or under heated conditions.

Next, the resin-bound peptide is washed at step (f) using the washing solution, which may comprise IPA, or optionally IPA and one of DMSO of EtOAc. For example, in specific embodiments, the resin-bound peptide may be washed 2 times in 10 volumes IPA/DMSO, wherein the concentration of DMSO may be 30% or more.

In embodiments, steps (c) through (f), including deprotecting, washing, coupling, and washing, may be repeated as many times as required to add sequential amino acids and complete the amino acid sequence of the peptide in a stepwise manner.

Once the desired amino acid sequence is achieved, the final product (the resin-bound peptide) is washed, dried, and then the peptide cleaved from the resin. In embodiments, the product of step (f) is further washed in IPA to remove trace amounts of reagents, such as DMSO or EtOAc. For examples, the product of step (f) may be washed 1-5 times with 10 volumes IPA to remove DMSO or EtOAc. Then, the resin-bound peptide is washed in step (g) with methanol, MTBE, or diethyl ether. For example, at step (g), the resin-bound peptide may be washed 1-5 times in 10 volumes of methanol, MTBE, or diethyl ether.

Next, the final product may be dried using methods known in the art. For example, the resin-bound product may be dried at 40° C. under vacuum. The resin-bound product may then be stored, for example at 2-8° C., or may be cleaved for further use.

The final peptide product may be cleaved from the resin using methods known in the art. For example, the peptide may be cleaved from the resin with an acidic cocktail of trifluoroacetic acid (TFA)/TIPS/DTT/water in the following ratio: (85% v: 5% v: 5% v: 5% w). Illustratively, the cleavage cocktail (10 mL/gm or more) is added to the resin and the suspension is stirred for 2-4 hr at room temp. The solution is filtered and the resin is washed with a small amount of cleavage solution. The cold MTBE or diethyl ether is added to the filtrate to obtain the precipitate. The resulting precipitate is centrifuged or filtered, e.g., through a sintered funnel. The residue is washed three times (3x) with MTBE, methanol, or diethyl ether, by centrifuging, decanting the filtrate, and filtering. The precipitated peptide is dried under vacuum, for example, at 40° C. overnight.

The skilled artisan will appreciate that various resins are known in the art and suitable for use in the present methods. In embodiments, the resin is a solid support resin, such as a bead. In embodiments, the resin may be selected from polystyrene-based resins, AAPPTec resin, Oxime resin, Wang resin, MBHA resin, Rink Amide resin, trityl based resins, Sieber resin, PAM (phenylacetamidomethyl) resin, PEG-based resins, (e.g., TentaGel®, Octagel™), and polyacrylamide resins (e.g., Amino-Li resin). Resins may further comprise linkers, which may include Wang linker, PAL, Rink Amide linker, 2-chlorotrityl linker, HMBA/HMPA linkers, HMBP linkers, PAM linkers, Merrifield linker, safety catch linkers, Rink Amide-AM, Rink Amide-MBHA, Sieber linker, Ramage linker, and the like. It is within the purview of the ordinary skilled person to select the appropriate resin/linker for synthesizing peptides according to the present methods.

In embodiments, the presently disclosed methods do not employ the use of DMF or DCM as a solvent in any step of the synthesis. In certain embodiments, the methods do not employ DMF or DCM as a solvent in any step during the synthesis of the first 25 amino acids of the peptide. After the first 25 amino acids are added to the peptide, in some embodiments, IPA is a major solvent for washing and deprotection steps, while coupling steps may employ DMSO, EtOAc, DMF, or DCM.

Steps of the present methods may be carried out at room temperature, or at elevated temperatures, depending on the particular amino acid and protecting group involved. As shown in Table 5, most Fmoc-amino acids can be dissolved effectively in a 0.2-0.3 M IPA-DMSO (70:30) mixture. However, certain Fmoc-amino acids, including Fmoc-Asn (Trt)-OH, Fmoc-Phe-OH, and Fmoc-His (Trt)-OH, have lower solubility in this mixture. To achieve their dissolution, a lower concentration (0.2 M) or IPA-DMSO (40:60) mixture or elevated temperature (40° C.) may be employed. Once solubilized, these amino acids can remain in solution even at room temperature.

TABLE 5
Solubility of Fmoc-amino acids in DMSO-IPA

| Fmoc-Amino Acid | Solubility in DMSO-IPA |
|---|---|
| Most Fmoc Amino Acids | Soluble in 0.4M IPA-DMSO (70:30) |
| Fmoc-Asn (Trt)-OH | Soluble in 0.2M IPA-DMSO (40:60) |
| Fmoc-Phe-OH | Soluble in 0.2M IPA-DMSO (40:60) |
| Fmoc-Arg(Pbf)-OH | Soluble at 0.4M IPA-DMSO (40:60) |

The utilization of isopropyl alcohol (IPA) as a green solvent in peptide synthesis offers a sustainable alternative to traditional methods, addressing environmental concerns associated with conventional solvents. The present disclosure evidences the successful implementation of IPA in various steps of peptide synthesis, from Fmoc group removal, various washes, and amino acid coupling. By incorporating IPA as a major solvent or co-solvent, this innovative approach not only promotes environmentally responsible practices in pharmaceutical manufacturing, but also demonstrates compatibility with both manual and automated peptide synthesizers with conventional heat and microwave low temperature. With its efficacy in producing a range of peptides, IPA could be employed as a solvent or co-solvent for synthesizing a wide variety of peptides, including GLP analogs like exenatide, liraglutide, semaglutide, and tirzepatide. IPA is a versatile and environmentally friendly solution for large-scale peptide production. The disclosed methods not only align with global sustainability efforts, but also showcase IPA's adaptability in diverse solvent mixtures tailored to specific synthesis requirements. The introduction of IPA in peptide synthesis represents a significant advancement in the field of peptide synthesis, promising efficient, sustainable, and scalable peptide production for the pharmaceutical industry.

### Examples

The following examples are given by way of illustration are not intended to limit the scope of the disclosure.

### Example 1. Synthesis of Exemplary Peptides

Table 6 sets forth exemplary peptides synthesized according to the presently disclosed methods. As used herein, when an amino acid abbreviation appears with a number above the amino acid, the number refers to the corresponding amino acid position in the final peptide product. The numbers are provided for convenience and the appearance or absence of such numbers in a sequence does not influence the amino acid sequence or the peptide indicated in such sequence. Certain peptides are protected, meaning that a protecting group is attached to a the indicated position. The artisan will recognize that a variety of protecting groups are well known, and alternative protecting groups may be suitable for a particular process or peptide.

TABLE 6
Exemplary Synthesized Peptides

| Peptide | Sequence | | Mass |
|---|---|---|---|
| No. | Identifier | Peptide | (Daltons) |
| 1 | SEQ ID NO: 1 | Fmoc-Gly¹-Pro-Ser-Ser-Gly-Ala-Pro-Pro-Pro- | 1073.82 |
| | | Ser¹⁰-NH₂ | |
| 2 | SEQ ID NO: 2 | Fmoc-Lys¹-Asn-Gly-Gly-Pro-Ser-Ser-Gly-Ala- | 1374.1 |
| | | Pro-Pro-Pro-Ser¹³-NH₂ | |
| 3 | SEQ ID NO: 3 | Fmoc-Ile¹-Ala-Gly-Gly-Pro-Ser-Ser-Gly-Ala-Pro- | 1316.2 |
| | | Pro-Pro-Ser¹³-NH₂ | |
| 4 | SEQ ID NO: 17 | Fmoc-Gly¹-Gly-Pro-Ser(tBu)-Ser(tBu)-Gly-Ala-Pro- | 1131.02 |
| | | Pro-Pro-Ser¹¹(tBu)-Resin | |
| 5 | SEQ ID NO: 4 | Fmoc-Ile¹-Glu-Trp-Leu-Lys-Asn-Gly-Gly-Pro- | 1916.2 |
| | | Ser-Ser-Gly-Ala-Pro-Pro-Pro-Ser¹⁷-NH₂ | |
| 6 | SEQ ID NO: 5 | Ac-Ser¹-Tyr-Ser-Leu-Glu-His-Phe-Arg-Trp-Gly- | 1630.93 |
| | | Leu-Pro-Val¹³-NH₂ | |
| 7 | SEQ ID NO: 6 | H-Val¹-Val-Glu-Glu-Ala-Glu-Asn⁷-NH₂ | 787.1 |
| 8 | SEQ ID NO: 7 | H-Gly¹-Pro-Ser-Ser-Gly-Ala-Pro-Pro-Pro-Ser¹⁰- | 874.74 (M + |
| | | NH₂ | Na)⁺ |
| 9 | SEQ ID NO: 8 | H-Leu¹-Ile-Ala-Gly-Gly-Pro-Ser-Ser-Gly-Ala- | 1206.65 |
| | | Pro-Pro-Pro-Ser¹³-NH₂ | |
| 10 | SEQ ID NO: 9 | Fmoc- Ser¹-Gly-Ala-Pro-Pro-Pro-Ser⁷-NH₂ | 831.7 |
| 11 | SEQ ID NO: 10 | Fmoc-Ser¹-Ser-Gly-Ala-Pro-Pro-Pro-Ser⁸-NH₂ | 919.4 |
| 12 | SEQ ID NO: 11 | Fmoc-Gly¹-Pro-Ser-Ser-Gly-Ala-Pro-Pro-Pro- | 1072.6 |
| | | Ser¹⁰-NH₂ | |
| 13 | SEQ ID NO: 12 | Fmoc-Gly¹-Gly-Pro-Ser-Ser-Gly-Ala-Pro-Pro-Pro- | 1131.45 |
| | | Ser¹¹-NH₂ | |
| 14 | SEQ ID NO: 13 | Fmoc-Ala¹-Gly-Gly-Pro-Ser-Ser-Gly-Ala-Pro- | 1201.65 |
| | | Pro-Pro¹¹-Ser-NH₂ | |
| 15 | SEQ ID NO: 14 | Fmoc-Ile¹-Ala-Gly-Gly-Pro-Ser-Ser-Gly-Ala-Pro- | 1315.1 |
| | | Pro-Pro-Ser¹³-NH₂ | |
| 16 | SEQ ID NO: 15 | Fmoc-Leu¹-Ile-Ala-Gly-Gly-Pro-Ser-Ser-Gly-Ala- | 1428.89 |
| | | Pro-Pro-Pro-Ser¹⁴-NH₂ | |
| 17 | SEQ ID NO: 16 | H-Trp¹-Leu-Ile-Ala-Gly-Gly-Pro-Ser-Ser-Gly- | 1391.70 |
| | | Ala-Pro-Pro-Pro-Ser¹⁵-NH₂ | |

### Example 2. Synthesis of Peptides 1-10

Each Peptide 1-10 is synthesized according to the following exemplary method.

Rink Amide AM resin or Rink Amide MBHA resin (1 gm), each possessing a loading capacity ranging from 0.3 mmol/g to 0.71 mmol/g preferably 0.5 mmol/gm, is used. A protected terminal amino acid is covalently bound to the resin.

De-protection is systematically conducted three times, each for durations of 5 minutes, 10 minutes, and 25 minutes, employing a solution comprising 20% piperidine, 1% DBU, and IPA-DMSO (v/v/w/v), with a preference for a DMSO concentration of 30% or higher. Subsequently, the resin is washed sequentially two times with IPA, and three times with IPA-DMSO (70:30 v/v); the removal of the Fmoc group is assessed and confirmed via the Kaiser test after thorough drainage.

### Condensation of Amino Acids

In a reactor, a solution containing Fmoc-AA-OH/HOBt (equivalent 2.0-4.0/equivalent 2.0-4.0) (1.5 mm scale) and IPA-DMSO (70/30, v/v) is combined, followed by the addition of DIC (2.0-4.0 equivalent). The reaction proceeds for a minimum of 1-2 hours with continuous stirring. Subsequently, the coupling efficiency is assessed using the Kaiser test following complete drainage.

### Re-Condensation and Acetylation

Following a 1-2 hour reaction period, the Ninhydrin test method monitors the reaction progress. In cases of incomplete reaction, Fmoc-AA-OH (equivalent: 1.0-2.0) in IPA mixture is combined with a solution of TBTU, HATU, or PyBOP (equivalent: 1.0-2.0) in DMSO or other solvent. DIPEA (equivalent: 2.0-4.0), or an IPA-DMSO solution (comprising more than 50% DMSO) is added for an additional reaction time of at least 1 hour, with monitoring through the Ninhydrin test method.

If the reaction is still incomplete after re-condensation, the amino remaining after the reaction is acetylated in 10% Ac₂O/10% Pyridine or DIEA/80% IPA-DMSO 70-30 (v/v/v) for 20-30 minutes and washed.

After the coupling of all amino acids in the sequence, the resin bound peptide is washed with MeOH, MTBE, or diethyl ether, dried in a vacuum, and weighed.

A cooled cocktail solution consisting of TFA/TIS/water/DTT (80-95% v/1-10% v/1-10% v/1-10% w) is employed to cleave the peptide from the resin. With continuous stirring, the cleavage reaction is conducted for 2-3 hours once the temperature stabilizes at 25° C.±5° C.

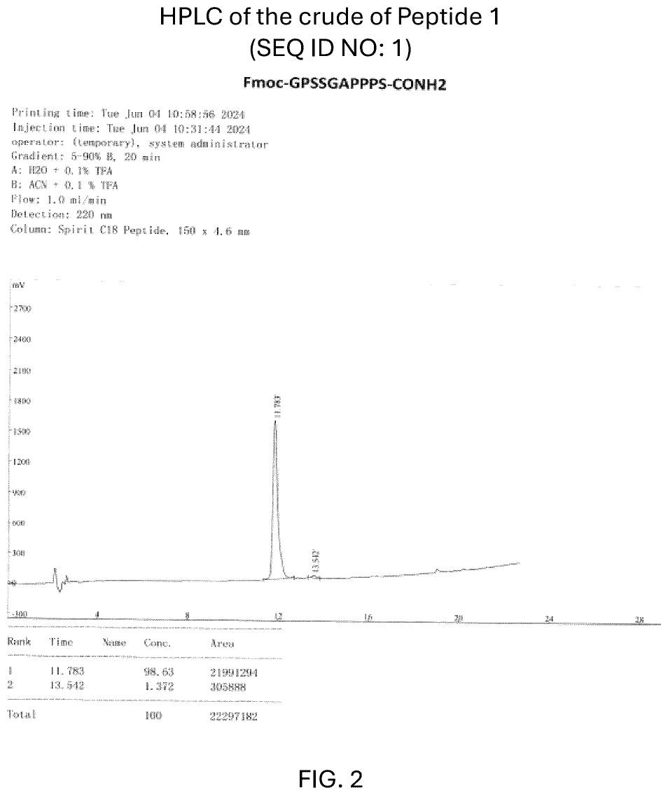

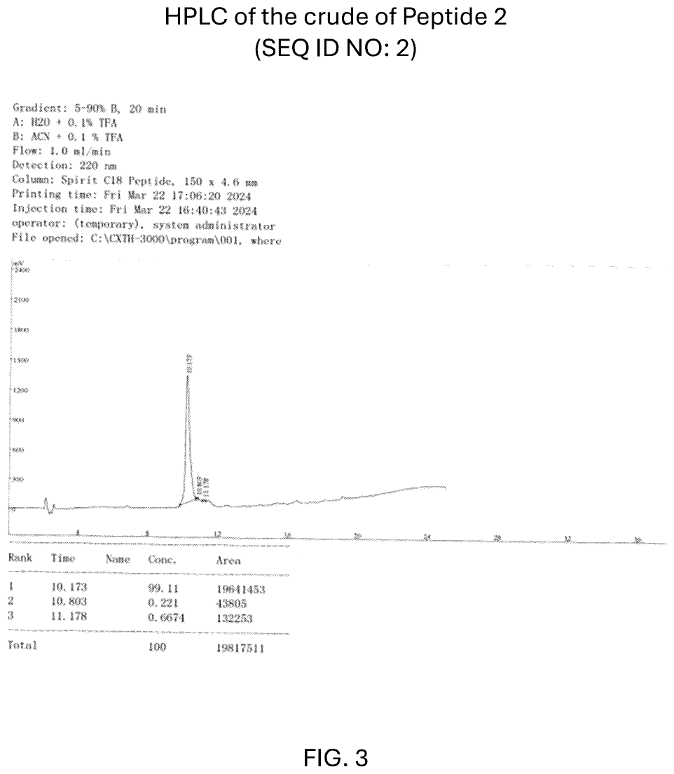

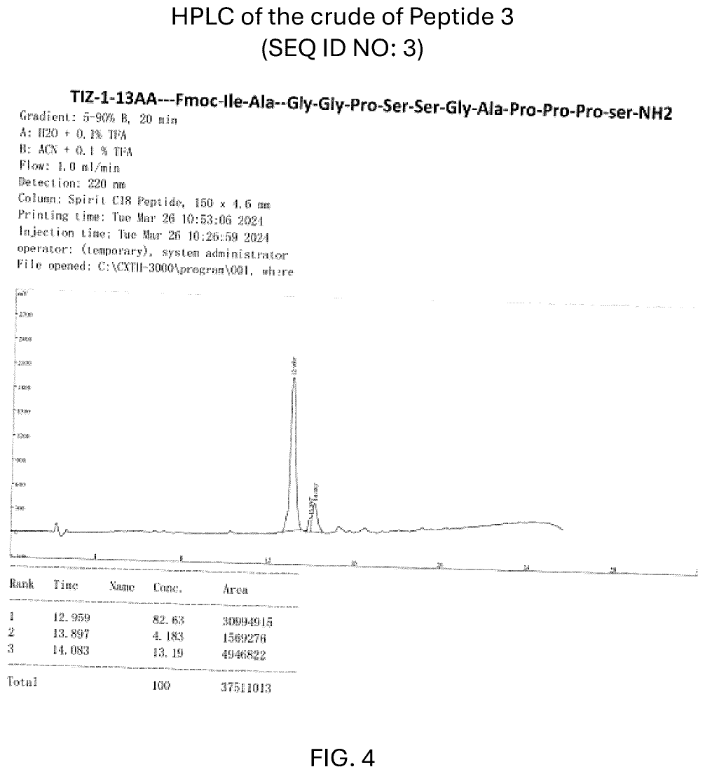

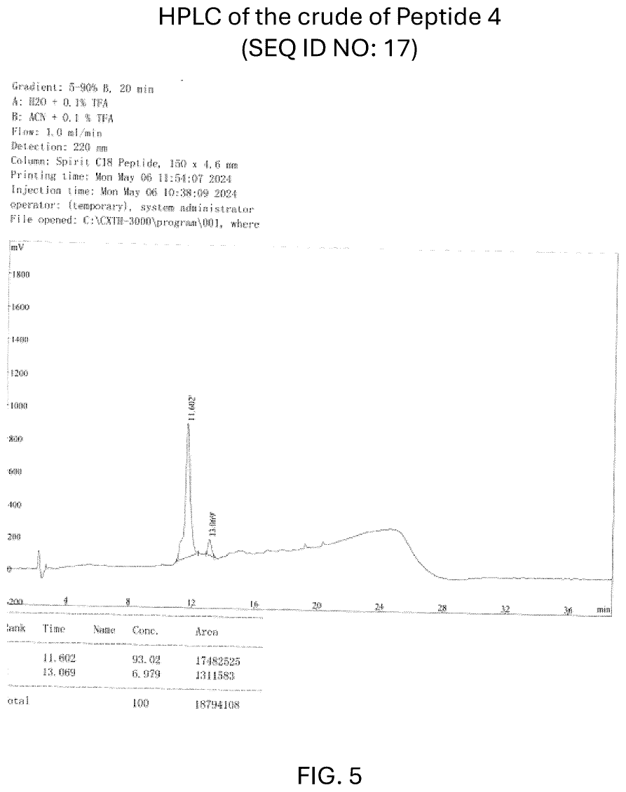

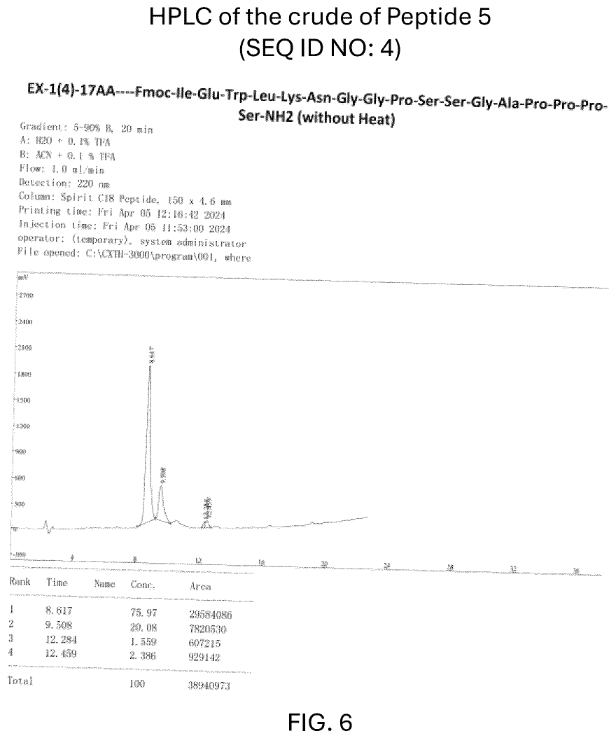

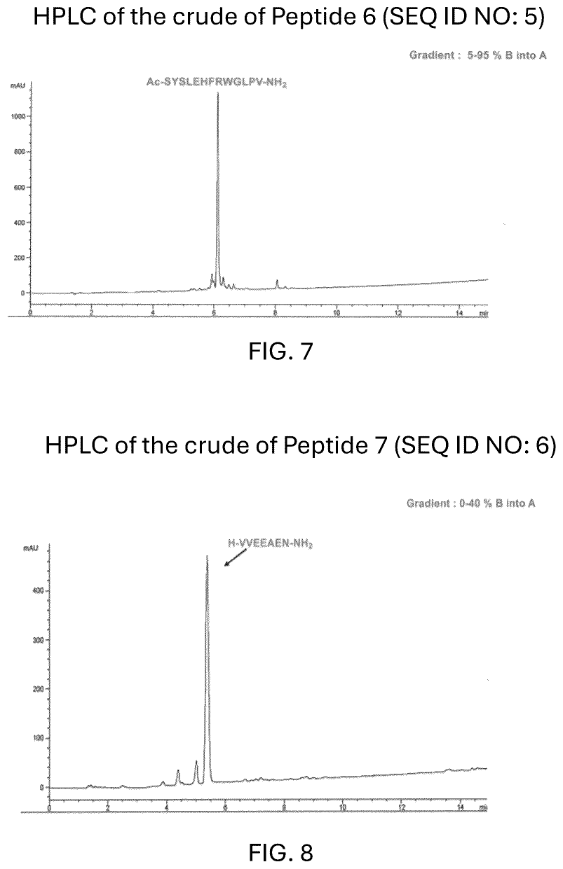

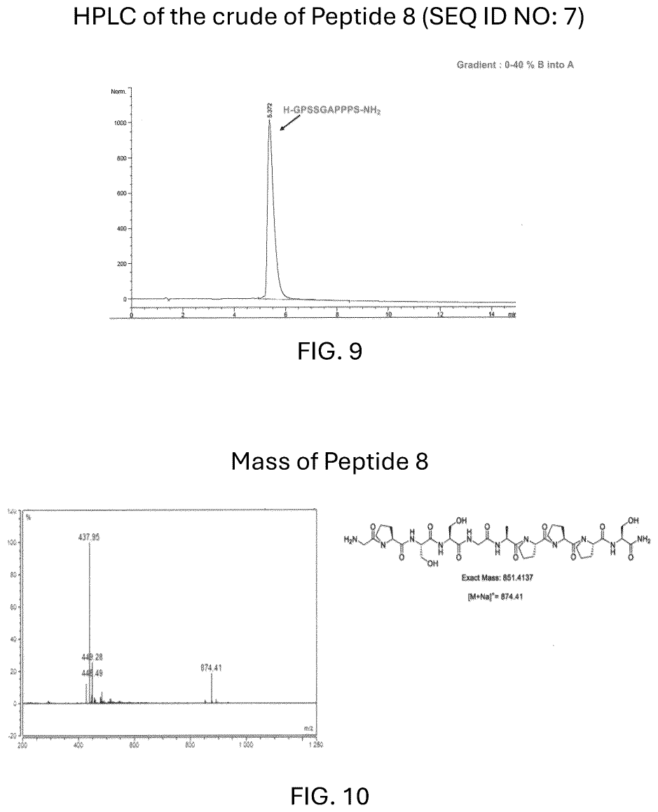

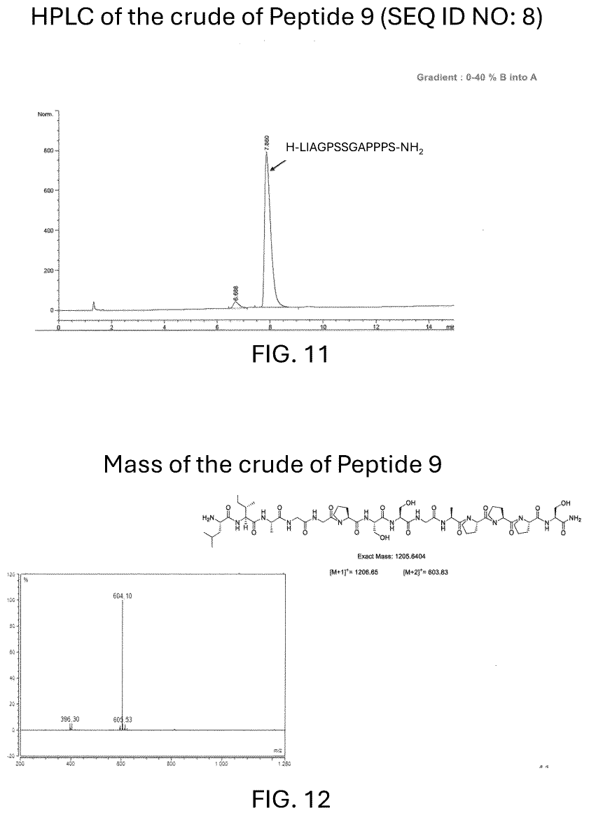

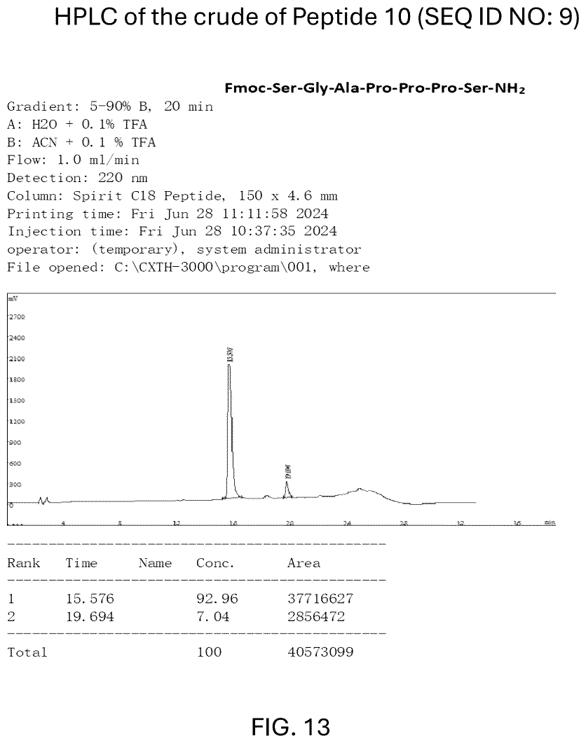

The condensed filtrate is poured into the cooled methyl MTBE, diethyl ether, isopropyl ether, or methyl ethyl ether (MEE) for precipitating the peptide; the filter cake is obtained by filtering or centrifuging, and is then thoroughly washed three times with cold MTBE, diethyl ether or isopropyl ether, or MEE. The crude polypeptide is transferred to a dryer and dried overnight under vacuum. Purity assessment is then performed using HPLC. See FIGS. 2-13 for respective HPLC and mass analyses of Peptides 1-10.

### Example 2. Synthesis of Peptides 11-17

Peptides 11-17 are synthesized according to the following exemplary method.

The OctaGel™ Rink amide resin exhibits a loading capacity of 0.45 mmol/g (1 gm). A protected terminal amino acid is covalently bound to the resin.

De-protection is systematically carried out three times, each lasting 5, 10, and 25 minutes, utilizing a solution containing 20% piperidine, 1% DBU, and IPA-DMSO in specified ratios, with a preference for a DMSO concentration of 30% or higher. Subsequently, the resin undergoes a sequential washing process involving two times with IPA, and three times with IPA-DMSO (70:30 v/v); confirmation of Fmoc group removal is verified through the Kaiser test post-drainage.

For amino acid condensation, a solution of Fmoc-AA-OH/HOBt (2.0-4.0 equivalents) preferably 3.0 equivalents (1.35 mmol scale) and IPA-DMSO (70/30, v/v) is introduced into a reactor, followed by the addition of DIC (2.0-4.0 equivalents). The reaction, facilitated by stirring, continues for at least 1-2 hours, with coupling efficacy evaluated post-draining using the Kaiser test.

After the completion of the total condensation reaction, the resin is washed with IPA/MTBE/Diethyl ether, then vacuum-dried and weighed. Subsequently, a chilled mixture of TFA/TIS/water/DTT (in specified proportions, 80-95% v/1-10% v/1-10% v/1-10% w) is employed to release the peptide from the resin, with the cleavage reaction sustained for 2-3 hours at a stabilized temperature of 25° C.±5° C. under continuous stirring.

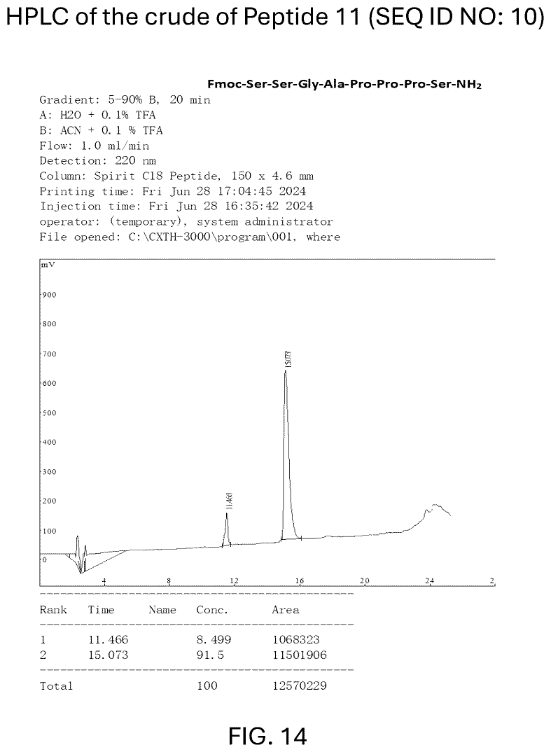

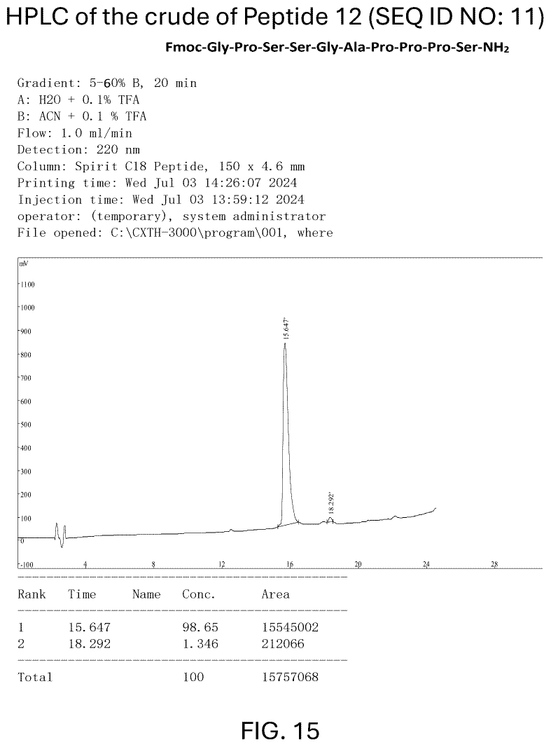

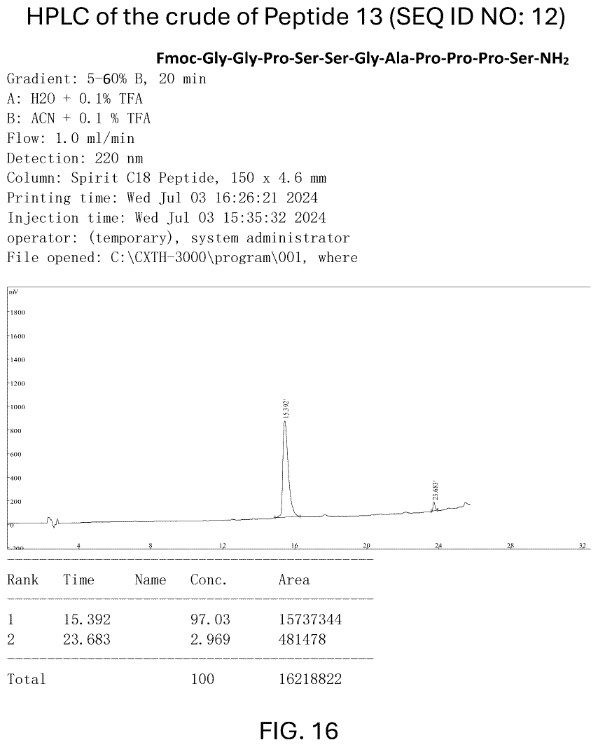

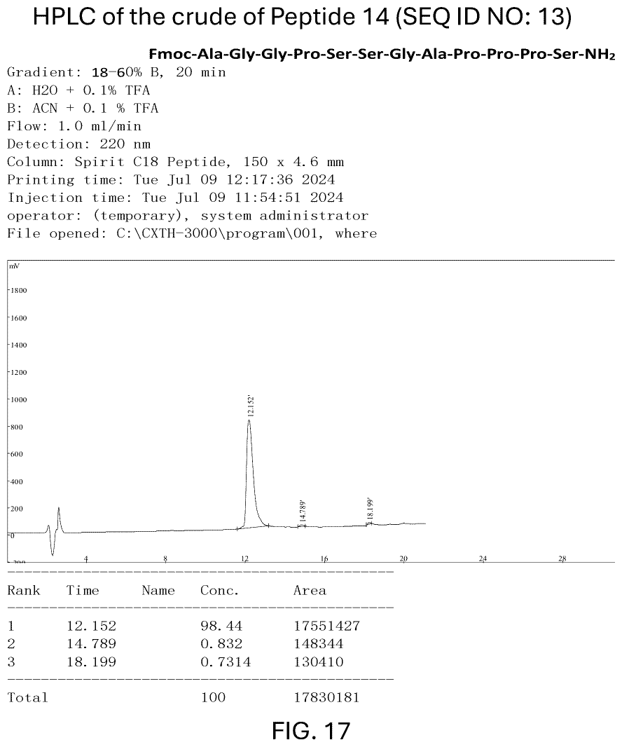

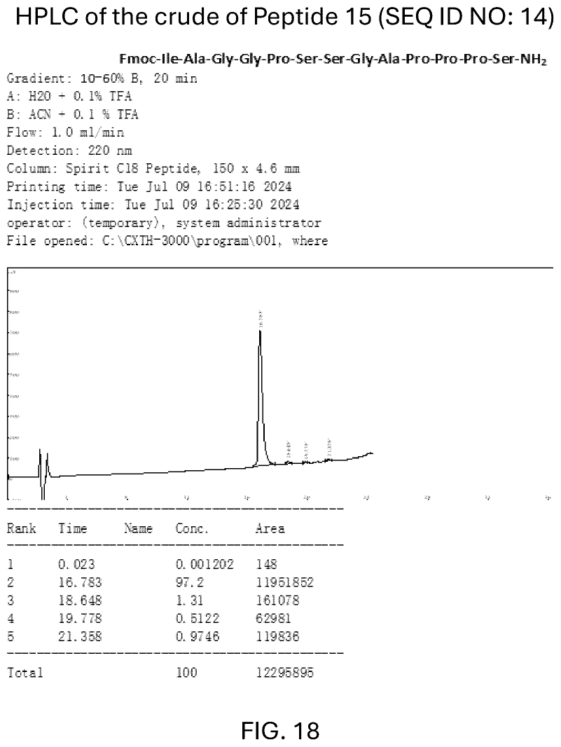

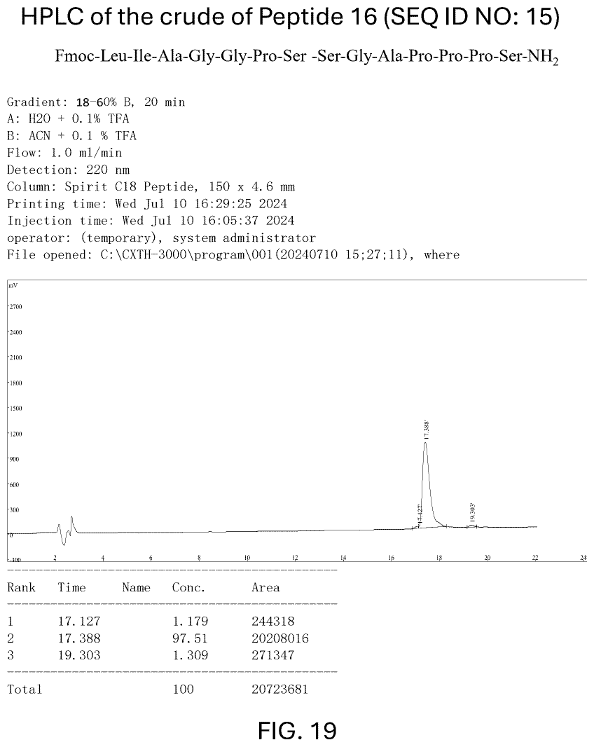

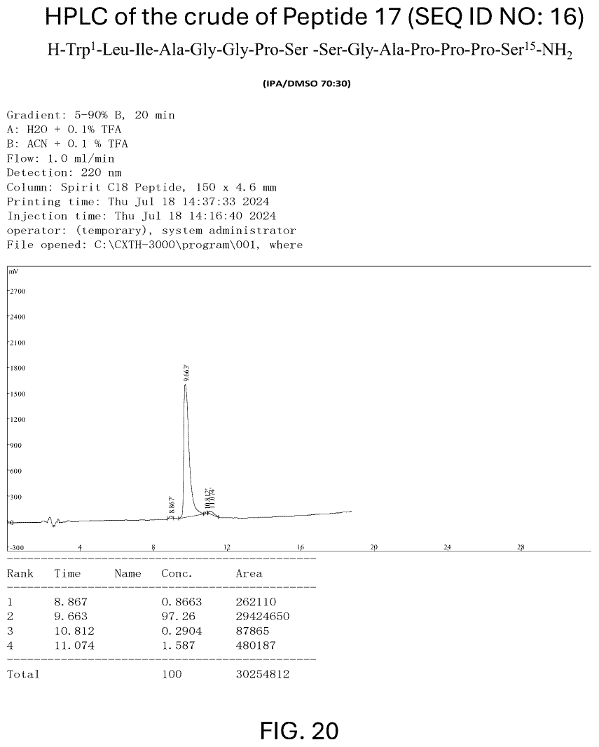

The resulting condensed filtrate undergoes sedimentation in cooled MEE, followed by the acquisition of the filter cake through filtration or centrifugation. The crude polypeptide is then transferred to a drying apparatus, where it is vacuum-dried for a minimum of 12 hours. Evaluation of purity can be conducted using HPLC. See FIGS. 14-20 for respective HPLC and mass analyses of Peptides 11-17.

### Example 3. Abbreviations

| | |
|---|---|
| Solid-phase peptide synthesis | SPPS |
| isopropyl alcohol | IPA |
| Solid-phase synthesis | SPS |
| N,N-dimethylformamide | DMF |
| Ethyl acetate | EtOAc |
| Dimethylsulfoxide | DMSO |
| 2-(1H-benzotriazole-1-yl)-1,1,3,3-tetram ethylaminium tetrafluoroborate | TBTU |
| Polystyrene | PS |
| Polyethyleneglycol | PEG |
| 17-Amino-10-oxo-3,6,12,15 tetraoxa-9-aza heptadecanoic acid | AEEA |
| (1-[Bis (dimethylamino) methylene]-1H-1,2,3-triazolo [4,5-b] pyridinium 3- | HATU |
| oxide hexafluorophosphate. | |
| Triethyl phosphate | TEP |
| Piperidine | Pip |
| Diisopropylcarbodiimide | DIC |
| Ethyl cyanohydroxy iminoacetate | Oxyma |
| Methyl-tert-butyl ether | MTBE |
| Trifluoroacetic acid | TFA |
| 1-Cyano-2-ethoxy-2 oxoethylideneaminooxy-tris-pyrrolidino-phosphonium | PyOxim |
| hexafluorophosphate | |
| Methyl ethyl ether | MEE |
| Mobile phase A | MPA |
| Mobile phase B | MPB |
| N,N-Diiso propylethylamine | DIEA |
| 9-fluorenylmethyloxycarbonyl | Fmoc |
| Active pharmaceutical | AP |
| Active pharmaceutical ingredient | API |
| (Benzotriazol-1-yloxy) tripyrrolidinophosphonium hexafluorophosphate | PyBOP |
| Chlorotrityl | CTC |
| Tert-butyloxycarbonyl | Boc |
| 2-(5-Norbornene-2,3-dicarboximido)-1,1,3,3-tetramethyluronium | TNTU |
| Tetrafluoroborates | |
| 6-Chloro-benzotri azole-1 yloxy-tris-pyrrolidinophosphonium hexafluoro | PyClock |
| phosphate | |
| Hydroxybenzo triazole | HOBt |
| High resolution mass spectrometry | HRMS |
| Liquid phase peptide synthesis | LPPS |
| Triisopropylsilane | TIPS |
| Dithiothreitol | DTT |
| High-Performance Liquid Chromatography | HPLC |

It is noted that the terms “substantially” and “about” may be utilized herein to represent the inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue. The term “substantially” is used herein also to represent the degree by which a quantitative representation may vary from a stated reference without resulting in a change in the basic function of the subject matter at issue. Thus, it is used to represent the inherent degree of uncertainty that may be attributed to any quantitative comparison, value, measurement, or other representation, referring to an arrangement of elements or features that, while in theory would be expected to exhibit exact correspondence or behavior, may in practice embody something less than exact.

It is noted that one or more of the following claims utilize the term “wherein” as a transitional phrase. For the purposes of defining the present technology, it is noted that this term is introduced in the claims as an open-ended transitional phrase that is used to introduce a recitation of a series of characteristics of the structure and should be interpreted in like manner as the more commonly used open-ended preamble term “comprising.”

It should be understood that where a first component is described as “comprising” or “including” a second component, it is contemplated that, in some embodiments, the first component “consists” or “consists essentially of” the second component. Additionally, the term “consisting essentially of” is used in this disclosure to refer to quantitative values that do not materially affect the basic and novel characteristic(s) of the disclosure.

It should be understood that any two quantitative values assigned to a property or measurement may constitute a range of that property or measurement, and all combinations of ranges formed from all stated quantitative values of a given property or measurement are contemplated in this disclosure.

While particular embodiments have been illustrated and described herein, it should be understood that various other changes and modifications may be made without departing from scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A method of solid-phase peptide synthesis (SPPS) of a peptide, the method comprising:
(a) coupling a protected amino acid to a resin;
(b) contacting the resin with a solvent comprising isopropyl alcohol (IPA) to swell the resin;
(c) deprotecting the protected amino acid with a deprotection solution comprising IPA to provide a deprotected amino acid;
(d) washing the product of step (c) with a washing solution comprising IPA;
(e) coupling an activated amino acid to the deprotected amino acid in the presence of a coupling solution comprising IPA;
(f) washing the product of step (e) with the washing solution comprising IPA;
(g) washing the product of step (f) with methanol, methyl-tert-butyl-ether (MTBE), or diethyl ether.

2. The method according to claim 1, wherein the protected amino acid of step (a) is a terminal amino acid of the peptide.

3. The method according to claim 2, wherein the terminal amino acid is protected with a protecting group is selected from 9-fluorenylmethoxycarbonyl (Fmoc), tert-butyloxycarbonyl (Boc), carboxybenzyl (CBZ), and 1-(4,4-dimethyl-2,6-dioxocyclohex-1-ylidene)ethyl(Dde).

4. The method according to claim 1, wherein the activated amino acid of step (e) is protected with a protecting group.

5. The method according to claim 4, wherein the activated amino acid is protected with a protecting group selected from 9-fluorenylmethoxycarbonyl (Fmoc), tert-butyloxycarbonyl (Boc), carboxybenzyl (CBZ), and 1-(4,4-dimethyl-2,6-dioxocyclohex-1-ylidene)ethyl(Dde).

6. The method of claim 4, wherein the method further comprises repeating steps (c) through (f) to add sequential amino acids to the peptide.

7. The method according to claim 1, wherein the solvent of step (b) further comprises dimethylsulfoxide (DMSO).

8. The method according to claim 7, wherein the solvent of step (b) comprises DMSO at a concentration of from about 30% to about 40% by volume.

9. The method according to claim 1, wherein the deprotection solution of step (c) further comprises piperidine and/or 1,8-Diazabicyclo[5.4. 0]undec-7-ene (DBU).

10. The method according to claim 9, wherein the deprotection solution of step (c) further comprises DMSO or ethyl acetate (EtOAc).

11. The method according to claim 1, wherein the washing solution of step (d) and/or step (f) further comprises DMSO or EtOAc.

12. The method according to claim 1, wherein the coupling solution of step (e) further comprises DMSO or EtOAc.

13. The method according to claim 1, further comprising drying the product of step (g).

14. The method according to claim 1, further comprising cleaving the peptide from the resin.

15. The method according to claim 1, wherein the resin is a solid support resin selected from Ramage resin, AAPPTec resin, Rink amide resin, MBHA resin, OctaGel™ resin, Oxime resin, Sieber amide resin, and PEG-based resin.

16. The method according to claim 1, wherein the method does not employ N,N-dimethylformamide (DMF) as a solvent in any step during synthesis of the first 25 amino acids of the peptide.

17. The method according to claim 1, wherein the method does not employ dichloromethane (DCM) as a solvent in any step during synthesis of the first 25 amino acids of the peptide.

18. The method according to claim 1, wherein the method is manual or automated.

19. The method according to claim 1, wherein the method is carried out with or without adding heat at any step.

20. The method according to claim 1, wherein step (c) and/or step (e) are carried out at room temperature, or at a temperature within about 5° C. of the boiling point of IPA.

21. The method according to claim 1, wherein IPA is a major solvent or a co-solvent in all washing steps.

22. The method according to claim 5, wherein after synthesis of the first 25 amino acids of the peptide, the coupling solution of step (e) further comprises EtOAc, DMF, or DCM.

23. A method of solid phase peptide synthesis (SPPS), wherein IPA is a major solvent or a co-solvent in all washing steps, and wherein the method does not employ N,N-dimethylformamide (DMF) as a solvent in any step.
